(19) United States
(12) Patent Application Publication
(10) Pub. No.: US 2024/0024653 A1
(43) Pub. Date: Jan. 25, 2024

(54) FLUID DELIVERY CONNECTOR PROTECTION DEVICES AND METHODS

(71) Applicants: Georgia Tech Research Corporation, Atlanta, GA (US); Children's Healthcare of Atlanta, Atlanta, GA (US)

(72) Inventors: Scott J. Hollister, Atlanta, GA (US); Ryan Akman, Atlanta, GA (US); Sarah Jo Tucker, Atlanta, GA (US); Joanna Kriegler Grossman, Atlanta, GA (US); Kishan Pithadia, Atlanta, GA (US); Adam S. Verga, Atlanta, GA (US)

(21) Appl. No.: 18/043,360

(22) PCT Filed: Sep. 3, 2021
(86) PCT No.: PCT/US2021/049111

§ 371 Date: Feb. 28, 2023

Related U.S. Application Data
(60) Provisional application No. 63/074,945, filed on Sep. 4, 2020.

(51) Int. Cl. A61M 39/20; A61M 39/18; A61M 39/14

(52) U.S. Cl. CPC A61M 39/20; A61M 39/18; A61M 39/14

(57) ABSTRACT

Systems, methods, and devices for prohibiting contaminants from entering a central venous catheter connection are disclosed. A device described herein includes a first body section defining a first internal cavity and a second body section defining a second internal cavity. The second body section can be hingeably connected to the first body section. A first tubing notch is positioned at an end of at least one of the first body section or the second body section. The device has an open configuration and a closed configuration, and wherein, in the closed configuration, the first internal cavity and the second internal cavity are adjacent to create a connector cavity configured to contain a first connector.

### CROSS-REFERENCE TO RELATED APPLICATIONS

This application claims priority, and benefit under 35 U.S.C. § 119(e), to U.S. Provisional Patent Application No. 63/074,945, filed 4 Sep. 2020, the entire contents of which are hereby incorporated by reference as if fully set forth below.

### FIELD OF THE DISCLOSURE

Embodiments of the present disclosure relate generally to protective covers for connectors of fluid delivery devices and, more particularly, to devices, systems, and methods to prevent contaminants from entering a central venous catheter connection.

### BACKGROUND

Central venous catheters (CVCs) and similar intravenous delivery devices can be used to deliver medicines to an individual for a prolonged period of time. For example, children with cancer and blood disorders may need CVCs to safely administer chemotherapy and other supportive medications, as well as to provide reliable vascular access for blood draws. There are many different types of CVCs—some are fully implanted under the skin and accessed with an external needle, others have tubing components that are suspended outside the chest wall, etc. Depending on the disease for which the patient is being treated, CVCs may be in place for just a few hours or for several years.

Although CVCs allow for easy venous access and safe medication administration in chronically ill and critically ill patients, infectious complications are common, affecting tens of thousands of people at United States hospitals annually. Certain products and designs have been implemented to decrease contamination of CVCs, for example to minimize the occurrence of central line-associated bloodstream infections (CLABSIs). Certain designs include caps that are screwed onto the end of a CVC connector and protective woven sleeves to cover a CVC connection. However, these prior designs have limitations—the caps can only be used when a CVC is not in use; the sleeves are difficult to remove when changing a CVC or a connection to the CVC. Further, even with these prior designs implemented, the rate of CLABSI among pediatric patients, for example, is still high—between 0.2-11 per 1000 central line days, depending on the individual patient's underlying condition and line characteristics. To this end, CLABSIs continue to be a substantial problem for pediatric oncology and bone marrow transplant patients and are responsible for excess morbidity and mortality.

### SUMMARY

Embodiments of the present disclosure address these concerns as well as other needs that will become apparent upon reading the description below in conjunction with the drawings. Briefly described, examples of the present disclosure relate generally to protective covers for connectors of fluid delivery devices and, more particularly, to devices, systems, and methods to prohibit contaminants from entering a central venous catheter connection.

An exemplary embodiment of the present invention provides a tubing connector protection device. The device can include a first body section defining a first internal cavity. The device can include a second body section defining a second internal cavity. The second body section can be hingeably connected to the first body section. The device can include a first tubing notch positioned at an end of at least one of the first body section or the second body section. The device can have an open configuration and a closed configuration. In the closed configuration, the first internal cavity and the second internal cavity can be adjacent to create a connector cavity configured to contain a first connector.

In any of the embodiments described herein, the first tubing notch can be positioned at a first end of the first body section. The second body section can include a second tubing notch positioned at a first end of the second body section. The second tubing notch can be positioned to align with the first tubing notch when the device can be in the closed configuration, thereby forming a first hole at the first end of the device configured to retain tubing for a catheter.

In any of the embodiments described herein, at least one of the first tubing notch or the second tubing notch can include a seal configured to close the first hole at the first end of the device.

In any of the embodiments described herein, the device can include a plug configured to close the first hole at the first end of the device.

In any of the embodiments described herein, the device can include a second tubing notch. The first tubing notch can be positioned at a first end of the first body section or the second body section, and the second tubing notch can be positioned at a second end of the second body section or the first body section, the first end being opposite from the second end along a length of the device.

In any of the embodiments described herein, the first tubing notch can be positioned at a first end of the first body section. The device can include a second tubing notch positioned at the first end of the second body section. The device can include a third tubing notch positioned at a second end of the first body section. The device can include a fourth tubing notch positioned at the second end of the second body section. The first tubing notch and the second tubing notch can be positioned to align when the device can be in the closed configuration to create a first hole. The third tubing notch and the fourth tubing notch can be positioned to align when the device can be in the closed configuration to create a second hole.

In any of the embodiments described herein, at least one of the first tubing notch or the second tubing notch can include a first seal configured to close the first hole at the first end of the device; and at least one of the third tubing notch or the fourth tubing notch can include a second seal configured to close the second hole at the second end of the device.

In any of the embodiments described herein, at least one of the first tubing notch or the second tubing notch can include a first seal configured to close the first hole at the first end of the device; or at least one of the third tubing notch or the fourth tubing notch can include a second seal configured to close the second hole at the second end of the device.

In any of the embodiments described herein, the device can include a plug configured to close at least one of the first hole or the second hole.

In any of the embodiments described herein, the device can include a first plug configured to close the first hole and a second plug configured to close the second hole.

In any of the embodiments described herein, the first body section and the second body section can form a capsular shape when the device can be in the closed configuration.

In any of the embodiments described herein, the first body section can include a flexible latch, and the second body section can include a latch peg. The flexible latch can be configured to detachably connect with the latch peg when the device can be in the closed configuration.

In any of the embodiments described herein, the first body section can include a latch finger configured to engage with a latch tab disposed on the second body section when the device can be in the closed configuration.

In any of the embodiments described herein, the first body section can include a plurality of latch fingers. Each latch finger can be configured to engage with one of a plurality of latch tabs disposed on the second body.

In any of the embodiments described herein, the first body section can include a first plurality of padding tabs disposed within the first internal cavity and configured to hold a first padding.

In any of the embodiments described herein, the second body section can include a second plurality of padding tabs disposed within the second internal cavity and configured to hold a second padding.

In any of the embodiments described herein, the first padding can be a sponge.

In any of the embodiments described herein, the sponge can include an antiseptic.

In any of the embodiments described herein, at least one of the first body section or the second body section can include a padding disposed within a respective internal cavity.

In any of the embodiments described herein, the device can include a sensor.

In any of the embodiments described herein, the sensor can be configured to detect contamination.

In any of the embodiments described herein, the sensor can be a colorimetric sensor.

In any of the embodiments described herein, the contamination can be bacteria.

In any of the embodiments described herein, the device can include a hinge pin. The first body section can include a first plurality of hinge knuckles; the second body section can include a second plurality of hinge knuckles positioned to engage with the first plurality of hinge knuckles in an alternating manner. The hinge pin can be configured to engage hinge holes disposed within the first and second pluralities of hinge knuckles.

In any of the embodiments described herein, the first body section and the second body section can form a single component that are connected via a flexible portion bendable to transition the device from the open configuration and the closed configuration.

In any of the embodiments described herein, the device can include protective padding surrounding the first body section and the second body section on an external face of the respective sections when the device can be in a closed configuration.

In any of the embodiments described herein, at least one of the first body section or the second body section can include a seal along an outer periphery of the respective body section. The seal configured to protect the connector cavity when the device can be in the closed configuration.

An exemplary embodiment of the present invention provides a system. The system can include a connector protection device. The connector protection device can include a first body section defining a first internal cavity. The connector protection device can include a second body section defining a second internal cavity, the second body section being hingeably connected to the first body section. The connector protection device can include a first tubing notch positioned at a first end of the connector protection device. The system can include a first connector having a first tube extending therefrom. When the connector protection device can be in a closed configuration, the first internal cavity and the second internal cavity can be adjacent to create a connector cavity configured to contain the first connector. When the connector protection device can be in a closed configuration, the first tube can extend through the first tubing notch.

In any of the embodiments described herein, the connector protection device can include a second tubing notch positioned at a second end of the connector protection device and sized to retain at least a portion of a fluid delivery tube.

In any of the embodiments described herein, the system can include a fluid delivery tube comprising a second connector. The connector cavity can be configured to contain a second connector connected to the fluid delivery tube.

In any of the embodiments described herein, a distance between the first tubing notch and the second tubing notch can be substantially the same as a length from a first end of the second connector to a second end of the first connector.

In any of the embodiments described herein, the connector protection device can include a first seal configured to close the first tubing notch and a second seal configured to close the second tubing notch.

In any of the embodiments described herein, the connector protection device can include a first seal configured to close the first tubing notch or a second seal configured to close the second tubing notch.

In any of the embodiments described herein, the connector protection device can include a first plug configured to close the first tubing notch and a second plug configured to close the second tubing notch.

In any of the embodiments described herein, the connector protection device can include a first plug configured to close the first tubing notch or a second plug configured to close the second tubing notch.

In any of the embodiments described herein, the first body section and the second body section can form a capsular shape when the device can be in the closed configuration.

In any of the embodiments described herein, the first body section can include a flexible latch, the second body section can include a latch peg, and the flexible latch can be configured to detachably connect with the latch peg when the device can be in the closed configuration.

In any of the embodiments described herein, the first body section can include a latch finger configured to engage with a latch tab disposed on the second body section when the device can be in the closed configuration.

In any of the embodiments described herein, the first body section can include a plurality of latch fingers. Each latch finger can be configured to engage with one of a plurality of latch tabs disposed on the second body.

In any of the embodiments described herein, the first body section can include a first plurality of padding tabs disposed within the first internal cavity and configured to hold a first padding.

In any of the embodiments described herein, the second body section can include a second plurality of padding tabs disposed within the second internal cavity and configured to hold a second padding.

In any of the embodiments described herein, the first padding can be a sponge.

In any of the embodiments described herein, the sponge can include an antiseptic.

In any of the embodiments described herein, at least one of the first body section or the second body section can include padding disposed within a respective internal cavity.

In any of the embodiments described herein, the connector protection device can include a sensor.

In any of the embodiments described herein, the sensor can be a contamination sensor.

In any of the embodiments described herein, the contamination sensor can be disposed within at least one of the first internal cavity or the second internal cavity and can be configured to detect a contamination.

In any of the embodiments described herein, the contamination sensor can be a colorimetric sensor.

In any of the embodiments described herein, the contamination can be bacteria.

In any of the embodiments described herein, the connector protection device can include a hinge pin. The first body section can include a first plurality of hinge knuckles; the second body section can include a second plurality of hinge knuckles positioned to engage with the first plurality of hinge knuckles in an alternating manner. The hinge pin can be configured to engage hinge holes disposed within the first and second pluralities of hinge knuckles.

In any of the embodiments described herein, the first body section and the second body section can form a single component that are connected via a flexible portion bendable to transition the device from an open configuration and the closed configuration.

In any of the embodiments described herein, the connector protection device can include a protective padding surrounding the first body section and the second body section on an external face of the respective sections when the device can be in a closed configuration.

In any of the embodiments described herein, the connector protection device can include a seal configured to close the first tubing notch.

In any of the embodiments described herein, the connector protection device can include a plug configured to close the first tubing notch.

In any of the embodiments described herein, the first connector can be a catheter connector, and the first tube is a catheter connection tube.

An exemplary embodiment of the present invention provides a method for sealing a connector end of a catheter tube. The method can include positioning a first connector on the connector end of the catheter tube within a connector protection device in an open configuration. The method can include positioning a catheter tube extending from the connector end within a first tubing notch of the connector protection device. The method can include hinging the connector protection device to a closed configuration such that the first connector can be enclosed within a connector cavity created by a first body section and a second body section of the connector protection device, wherein hinging the connector protection device seals the first connector within the connector protection device to prevent contamination at the first connector.

In any of the embodiments described herein, the method can include plugging a second tubing notch of the connector protection device, the second tubing notch positioned at an end opposite the first tubing notch on the connector protection device.

In any of the embodiments described herein, the method can include attaching a second connector to the first connector. The method can include positioning a fluid delivery tube extending from the second connector within a second tubing notch of the connector protection device at an end opposite the first tubing notch. Hinging the connector protection device closed can cause the second connector to be sealed within the connector cavity.

In any of the embodiments described herein, the method can include latching the connector protection device, wherein the latching can include connecting a flexible latch on the first body section to a latch peg on the second body section.

In any of the embodiments described herein, hinging the connector protection device to the closed configuration can cause a latch finger on the first body section to engage with a latch tab on the second body section.

In any of the embodiments described herein, hinging the connector protection device to the closed configuration can cause a plurality of latch fingers on the first body section to engage with a respective plurality of latch tabs on the second body section.

In any of the embodiments described herein, the method can include comprising adding an antiseptic.

In any of the embodiments described herein, the method can include adding the antiseptic can include adding the antiseptic a first padding positioned within at least one of the first body section or the second body section.

### BRIEF DESCRIPTION OF THE FIGURES

Reference will now be made to the accompanying figures and diagrams, which are not necessarily drawn to scale, and wherein:

FIG. 1 is an upper perspective view of an example connector protection device, according to some embodiments of the present disclosure;

FIG. 2A is an exploded view of an example connector protection device shown from the external face(s) of the device, according to some embodiments of the present disclosure;

FIG. 2B is an exploded view of an example connector protection device shown from the internal face(s) of the device, according to some embodiments of the present disclosure;

FIG. 3A is a perspective view of an example single-tube connector protection device in an open configuration, according to some embodiments of the present disclosure;

FIG. 3B is a perspective view of an example multi-tube connector protection device in an open configuration, according to some embodiments of the present disclosure;

FIG. 4A is a perspective view of an example connector protection device having a padding in the internal cavities of the one or more body sections, according to some embodiments of the present disclosure;

FIG. 4B is a perspective view of an example connector protection device in a closed configuration, according to some embodiments of the present disclosure;

FIG. 5 is an end perspective view of an example connector protection device, according to some embodiments of the present disclosure; and

FIG. 6 is a flowchart of an exemplary method for sealing a connector end of a catheter tube, according to some embodiments of the present disclosure.

### DETAILED DESCRIPTION

Although certain embodiments of the disclosure are explained in detail, it is to be understood that other embodiments are contemplated. Accordingly, it is not intended that the disclosure is limited in its scope to the details of construction and arrangement of components set forth in the following description or illustrated in the drawings. Other embodiments of the disclosure are capable of being practiced or carried out in various ways. Also, in describing the embodiments, specific terminology will be resorted to for the sake of clarity. It is intended that each term contemplates its broadest meaning as understood by those skilled in the art and includes all technical equivalents which operate in a similar manner to accomplish a similar purpose.

It should also be noted that, as used in the specification and the appended claims, the singular forms “a,” “an” and “the” include plural references unless the context clearly dictates otherwise. References to a composition containing “a” constituent is intended to include other constituents in addition to the one named.

Ranges may be expressed herein as from “about” or “approximately” or “substantially” one particular value and/or to “about” or “approximately” or “substantially” another particular value. When such a range is expressed, other exemplary embodiments include from the one particular value and/or to the other particular value.

Herein, the use of terms such as “having,” “has,” “including,” or “includes” are open-ended and are intended to have the same meaning as terms such as “comprising” or “comprises” and not preclude the presence of other structure, material, or acts. Similarly, though the use of terms such as “can” or “may” are intended to be open-ended and to reflect that structure, material, or acts are not necessary, the failure to use such terms is not intended to reflect that structure, material, or acts are essential. To the extent that structure, material, or acts are presently considered to be essential, they are identified as such.

It is also to be understood that the mention of one or more method steps does not preclude the presence of additional method steps or intervening method steps between those steps expressly identified. Moreover, although the term “step” may be used herein to connote different aspects of methods employed, the term should not be interpreted as implying any particular order among or between various steps herein disclosed unless and except when the order of individual steps is explicitly required.

The components described hereinafter as making up various elements of the disclosure are intended to be illustrative and not restrictive. Many suitable components that would perform the same or similar functions as the components described herein are intended to be embraced within the scope of the disclosure. Such other components not described herein can include, but are not limited to, for example, similar components that are developed after development of the presently disclosed subject matter. Additionally, the components described herein may apply to any other component within the disclosure. Merely discussing a feature or component in relation to one embodiment does not preclude the feature or component from being used or associated with another embodiment.

To facilitate an understanding of the principles and features of the disclosure, various illustrative embodiments are explained below. In particular, the presently disclosed subject matter is described in the context of connector protection devices that can protect an end of central venous catheters (CVCs), either when connected to fluid delivery tubes or when not connected to fluid delivery tubes. For example, in certain scenarios, a patient may have a CVC that is inserted into a blood vessel but that is not connected to an external fluid delivery device (e.g., a device providing medicine). In these cases, the systems and devices described herein provide a solution to cap off the end of the disconnected CVC, thereby preventing bacteria and other contaminants from entering the end of the CVC and gaining access to the interior of the CVC lumen. In other scenarios, the CVC can be connected to an external fluid delivery device, and the systems and devices described herein provide a solution to seal off contaminants from entering the patient's tubing at the tubing connection. The present disclosure, however, is not limited to CVC connections and can be applicable in other contexts. For example, the embodiments of the systems and methods described herein may improve other tubing connections wherein care should be taken to ensure contaminants do not encroach the connection. Accordingly, when the present disclosure is described in the context of connector protection devices that can protect an end of CVCs, it will be understood that other embodiments of the present invention may be configured for use with other devices having tubing through which fluids are transferred into or out of the body, such as intravascular lines, including but not limited to central venous access devices, implantable ports, dialysis catheters, and so forth. It should be appreciated that as used herein, the term “fluids” may include medicines, blood, liquid nourishment, gases such as oxygen, and anything else with fluidic properties. For clarity, there may be times at which no fluid is flowing through the tubing.

As stated above, tubing-site infections such as central line-associated bloodstream infections (CLABSIs) are common complications, affecting tens of thousands of people at U.S. hospitals alone. Prior designs that included protective caps that threaded onto the end of a CVC are only helpful in the cases where the CVC is not connected to other tubing. Other designs, such as woven sleeves that slide over the CVC or other intravenous (IV) device, are difficult to remove and/or replace when a fluid delivery tube is connected to the CVC. The disclosure provides an outer enclosure (i.e., a CVC connection enclosure) that can reduce environmental microbial bioburden that contaminates the line tubing at connection sites, which can in turn lead to decreased occurrence of infections like CLABSI. The solutions described herein can include devices that can clasp over the CVC, whether the CVC is connected to a fluid delivery tube or not. Some embodiments of device 100 may be configured for single-use, while others are configured for multi-use.

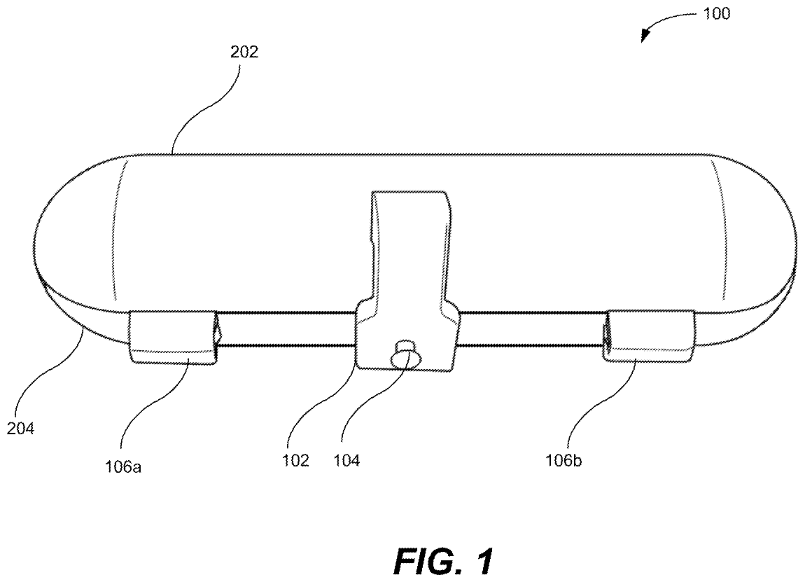

Reference will now be made to the accompanying figures and diagrams, which provide examples of connector protection devices, systems, and methods. FIG. 1 is an upper perspective view of an exemplary connector protection device 100, according to some embodiments of the present disclosure. The connector protection device 100 can also be referred to as a “device 100” throughout this disclosure. FIG. 1 shows device 100 in a closed configuration. As will be described in greater detail below, the device 100 can transition between a closed configuration and an open configuration. In the open configuration, the internal cavities are exposed such that one or more connectors can be inserted into and/or removed from the device; in the closed configuration, the one or more connectors are housed within the device. The device 100 can include two body sections, a first body section 202 and a second body section 204 that are hingeably connected to one another. As will be appreciated, terms such as “first” and “second” in this disclosure do not refer to position, placement, and/or sequence of a particular component but are instead used to differentiate components from each other. Further, if a feature is described herein as being positioned/disposed on a first component, nothing prevents the feature from being positioned/disposed on a second component. To illustrate without limitation, if reference is made to a flexible latch 102 being disposed on a first body section 202, the flexible latch 102 can equally be disposed on the second body section 204; the latch peg 104 that corresponds to the flexible latch 102 will be on the opposite body section as the flexible latch 102.

Referring to the material composition of the device 100 (e.g., the material composition of the first body section 202 and/or second body section 204), the materials used to manufacture components of the device 100 can include polymers, for example plastics and/or resins. Other embodiments of device 100 comprise components composed of materials including metals, ceramics, composite materials, naturally and biologically occurring and derived materials, including materials that are recyclable and/or compostable, for example pressed organic fiber. The material used can be non-porous such that the device can be sterilized or sanitized, such as by wiping with disinfectant, for example betadine, chlorohexidine gluconate, alcohol, etc. Non-porous materials also can enable the device 100 to tolerate bodily and other fluids, such as medications. Alternative embodiments of device 100 comprise components composed of materials that are porous. Such components may be treated for hydrophobicity and sterility and/or coated with a non-porous film. It is contemplated that the components of the device 100 can be injection molded, compression molded, extruded, shrink wrapped, casted, machined, printed, and the like.

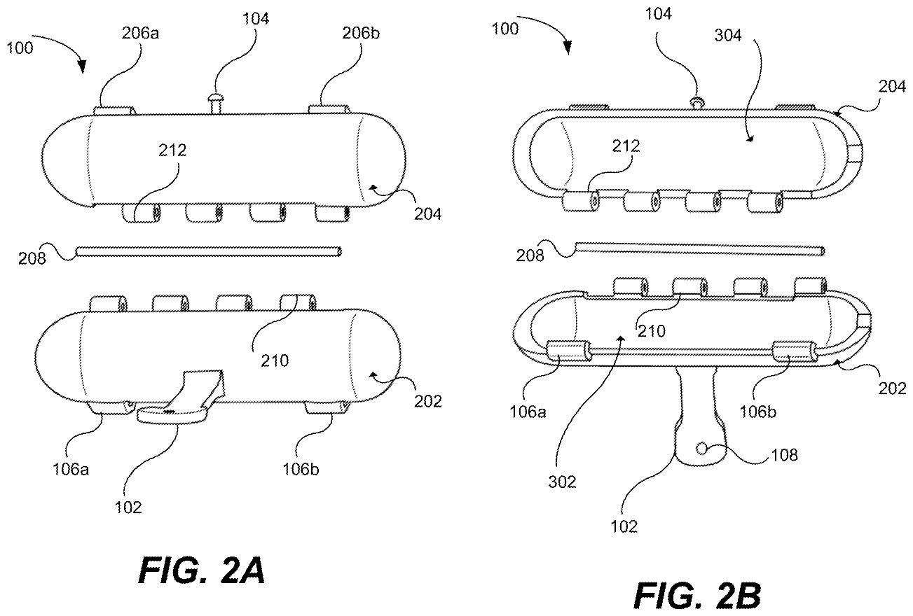

Referring again to FIG. 1, the device 100 can include the flexible latch 102 referenced above. The flexible latch 102 can extend from a body section, e.g., the first body section 202 as shown. The flexible latch 102 and the first body section 202 can be manufactured as a unitary component, for example by manufacturing the flexible latch 102 with a more flexible resin than the body section. The flexible latch 102 can also be a separate component that is connected to the first body section 202, for example via an adhesive or other attachment, such as a fastener, which could include a screw, nail, or pin. When the device is in a closed configuration, the flexible latch 102 can be moved such that a latch hole 108 (see FIG. 2B) on the flexible latch 102 securely receives a latch peg 104 on the other body section (e.g., the second body section 204). Flexible latch 102 has a length that ensures first and second body sections 202 and 204 are retained in mating engagement when latch peg 104 is inserted through latch hole 108.

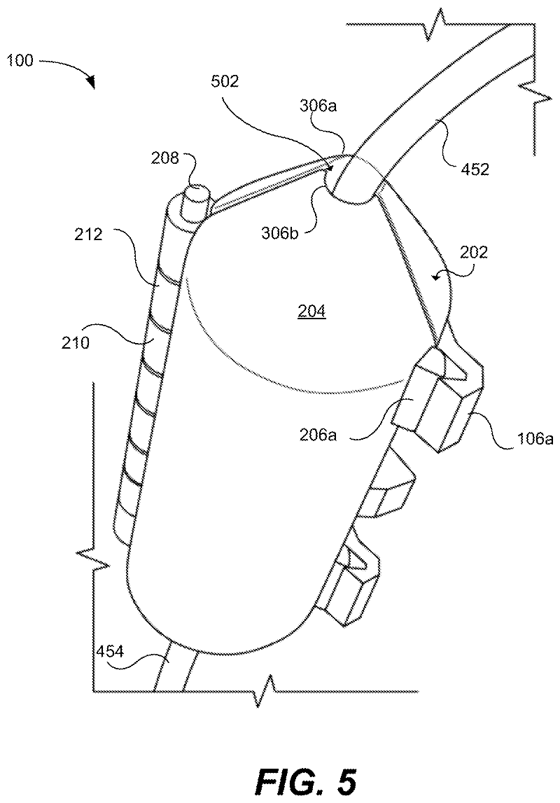

Alternatively or in addition to a flexible latch 102, the device 100 can include one or more other connection means to ensure that the device remains in a closed configuration. For example, the device 100 can include one or more latch fingers, shown in FIG. 1 as a first latch finger 106a and a second latch finger 106b (collectively referred to as latch finger(s) 106a,b). A latch finger 106a,b can be a curved flange projecting from the body section (e.g., the first body section 202) that engages with a latch tab on the other body section (e.g., the second body section 204). FIGS. 2A and 2B provide an example of a first latch tab 206a and a second latch tab 206b (collectively referred to as latch tab(s) 206a,b). FIG. 5 provides a view of how latch fingers 106a,b can engage with latch tabs 206a,b to ensure the device 100 maintains a closed configuration. It is contemplated that the device 100 can include any combination or quantity of flexible latches 102 and/or latch fingers 106a,b. For example, the device 100 can have one flexible latch 102 and two latch fingers 106a,b, as shown in FIG. 1; the device 100 can have no flexible latch 102 and one or more latch fingers 106a,b; the device can have no latch fingers 106a,b, and one or more flexible latches 102; and the like. Alternative embodiments of device 100 comprise other connection means to ensure that the device remains in a closed configuration, for example adhesives, hook-and-loop fasteners such as Velcro, magnets, and self-sealing attachments, in addition to or instead of any combination or quantity of flexible latches 102 and/or latch fingers 106a,b. In some embodiments, latch fingers 106a,b may be flexible, while in others they may be more rigid in order to provide a friction fit with latch tabs 206a,b.

FIG. 2A is an exploded view of an example connector protection device 100 shown from the external face(s) of the device, according to some embodiments of the present disclosure; FIG. 2B is an exploded view of an example connector protection device 100 shown from the internal face(s) of the device, according to some embodiments of the present disclosure. As stated above, the first body section 202 can be hingeably connected to the second body section 204 so that the device can transition between a closed configuration and an open configuration. In some examples, the device 100 can include a hinge pin 208 that holds together the two body sections. The first body section 202 can include a first set of hinge knuckles 210, and the second body section 204 can include a second set of hinge knuckles 212. When hinged knuckles 210 and 212 are interlaced such that their respective holes are axially aligned, the hinge pin 208 can be inserted into the holes of the respective hinge knuckles 210,212 to hold the body sections together, like in a door hinge. This configuration can be established during assembly. It can be appreciated that hinge pin 208 and knuckles 210,212 can be configured to prevent the hinge pin 208 from unintentionally disengaging, such as due to use, gravity, vibration, etc. This could be achieved for example by hinge pin 208 comprising threads or flared ends. In other examples, the hinge can be a flexible portion of the material between the first body section 202 and the second body section 204 that is bendable to transition the device between the open configuration and the closed configuration. For example and not limitation, the first body section 202 and the second body section 204 can be printed with a resin and connected by a section of resin that is substantially thinner than the material forming the body sections—the body sections can hinge about that thinner section. Some embodiments of device 100 can include other types of flexible hinges, including but not limited to flexible hinges. The flexible hinges can also include compliant mechanisms that maintains its position in the open or closed configuration, including for example the addition of springs to maintain position of the body sections.

It is contemplated that the device 100 can include a protective padding surrounding the first body section 202 and the second body section 204 on an external face of the respective sections. Referring to FIG. 2A to illustrate, the external faces shown can include a protective padding, such as foam, rubber, hydrophobic fabric, and the like that makes the device softer. This can be beneficial in the case, for example, the patient is a pediatric patient and the device 100 is attached to a CVC. The protective padding covering the external faces can help to soften the device so that it does not cause discomfort to the patient if the patient lies upon the device 100, for example. It is also contemplated that the shape of the device can also provide comfort for the user. The first body section 202 and the second body section 204 can form a capsular shape when the device is in the closed configuration (see FIG. 1). This shape can further prevent discomfort for the patient, since the CVCs can be worn for considerable lengths of time. In alternative embodiments, the first body section 202 and the second body section 204 form cylindrical, spherical, and prism shapes.

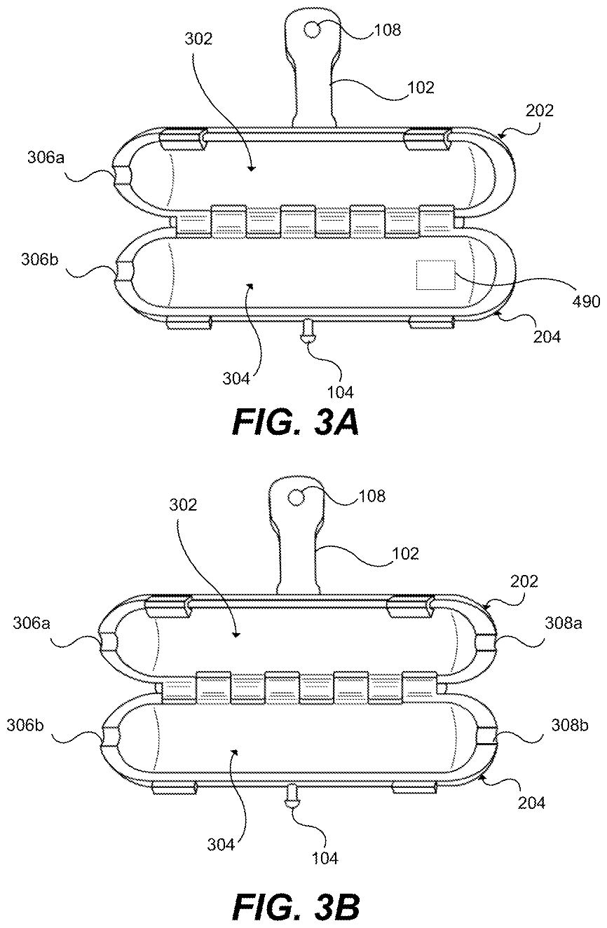

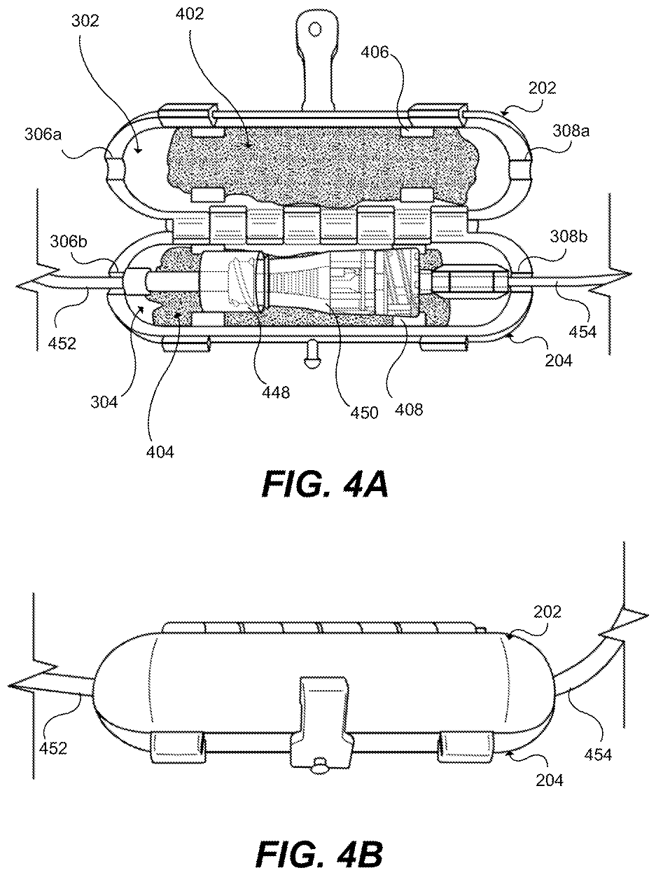

FIG. 3A is a perspective view of an example single-tube connector protection device 100 in an open configuration, according to some embodiments of the present disclosure; FIG. 3B is a perspective view of an example multi-tube connector protection device 100 in an open configuration, according to some embodiments of the present disclosure. The respective body sections can include internal cavities that provide space for the connectors of tubing (e.g., CVC tubes, fluid delivery tubes, etc.). For example, the internal face of the first body section 202 can define a first internal cavity 302, and the internal face of the second body section 204 can define a second internal cavity 304. When the device 100 is in a closed configuration the first internal cavity 302 and the second internal cavity 304 can be adjacent to create a connector cavity configured to contain a catheter connector (e.g., catheter connector 448 shown in FIG. 4A) and other connectors (e.g., fluid delivery tube 450 shown in FIG. 4A).

The device 100 can have one or more features that enable tubing to pass through the closed device 100 when a connector is inserted within the connector cavity. These features are referred to herein as tubing passageways. FIG. 3A provides an example device 100 that can be considered a single-tube device, as in only one end of the device 100 includes a tubing passageway. As can be seen, the device can have one or more tubing notches at one end of the device 100. In one example, the device can include a first tubing notch 306a formed in the first body section 202 and a second tubing notch 306b formed in the second body section 204 (as shown in FIG. 3A). The first tubing notch 306a and second tubing notch 306b can be positioned to align with each other when the device is in the closed configuration. In this example, once the device is closed, a tubing hole is created to serve as the tubing passageway for the tubing to pass through (see for example the tubing hole 502 in FIG. 5). As shown in FIG. 3A, when there is a first tubing notch 306a that is positioned to align with a second tubing notch 306b, the notches can be half-circular in shape such that, when they meet, they can form a circular hole at the end of the device 100. In this embodiment, the combination of the first tubing notch 306a and the second tubing notch 30b is the “tubing passageway” that enables tubes to pass through the closed device 100. It is also contemplated that, instead of having two notches that align when the device 100 is closed (e.g., one notch on the first body section 202 and one notch on the second body section 204), there is only a single notch on one of the first or the second body section. The one notch can be shaped to enable a tube to be retained by and/or pass through the device, serving on its own as a tubing passageway; for example, the singular notch (306a or 306b) can take a more circular shape or any other shape to accommodate a tube passing through the connector cavity. In some embodiments, device 100 comprises more than two tubing passageways, and the device is not limited to having tubing passageways only at the ends of the device.

Referring to FIG. 3B, some examples of the device 100 can include a second tubing passageway. For example, the single-tube embodiment in FIG. 3A can be used to protect the end of a CVC that is disconnected from external tubing; the multi-tube embodiment in FIG. 3B can be used to protect the end of a CVC that is disconnected from external tubing and/or can be used to protect a catheter connector 448 that is connected to a delivery connector 450. In this example, the device 100 can include a first tubing notch 306a and/or a second tubing notch 306b (as in FIG. 3) at one end of the device 100 and one or more notches at the other end of the device 100. For example, the device can include a third tubing notch 308a disposed on the first body section 202 and a fourth tubing notch 308b disposed on the second body section 204 (as shown in FIG. 3B). The first tubing notch 308a and second tubing notch 308b can be positioned to align with each other when the device is in the closed configuration, forming a tubing hole and serving as the first tubing passageway. In this example, once the device is closed, a second tubing hole 502 is formed on the device at an end opposite the first tubing hole, and the second tubing hole 602 can serve as the second tubing passageway. It is also contemplated that, for the second tubing passing way, instead of having two notches that align when the device 100 is closed (e.g., one notch on the first body section 202 and one notch on the second body section 204), there is only a single notch on one of the first or the second body section (as described above with reference to FIG. 3A and the notches at the first end of the device 100).

Some embodiments of device 100 can include a gasket disposed on one or both of the first body section 202 or second body section 204. Such gasket may be disposed on some or all of the possible points of contact between first body section 202 and second body section 204. In some embodiments, a gasket is disposed on the tubing passageway (e.g., on or around the notches), creating a secure encompassment of any tubing passing through a tubing passageway. In some embodiments, a gasket may be built-in component of device 100 secured to one or both body sections, or the gasket can be a separate component. In some examples, the gasket can be applied to the device by a coating, which can include but is not limited to a coating manufactured by dipping the body section into a rubber or similar material. To illustrate the gasket by referring to FIG. 5, the opening between the tube (e.g., catheter tube 452 as shown) and the tubing hole 502 can include a gasket or other enclosure to prevent fluid and/or contaminants from entering the tubing passageway.

It is contemplated that any of the notches (e.g., any of notches 306a, 306b, 308a, and 308b) can include one or more seals to prevent contamination from entering the connector cavity when the device is closed. The seal can be a silicon, rubber, etc. seal that is positioned within one or more of the notches such that, when there is no tubing passing through the notch(es) at that end of the device, the device is sealed from contamination. The seal should enable the tubing to pass through the one or more notch(es) when needed. In other examples, the seal(s) can be flanges extending from the body sections that are formed from the material that creates the body sections. For example, in the case that the body sections are printed, additional, thin material can be printed around the one or more notches to seal off that end of the device when no tubing is present. In the case that the device 100 is a single-tube device (e.g., FIG. 3A), the seal can be on the one end of the device that includes the one or more notches. In the case that the device 100 is a multi-tube device (e.g., FIG. 3B), the seal can be on one end or both ends of the device.

In some example devices 100, any of the notches (e.g., any of notches 306a, 306b, 308a, and 308b) can include one or more plugs to prevent contamination from entering the connector cavity when the device is closed. Unlike the seals described above, which are integrated within or proximate the one or more notch(es), the plugs can be inserted into and removed from the tubing passageway(s). For example, the plug can be a separate silicon, rubber, etc. plug that can be inserted into a tubing passageway to prevent contamination from entering that section of the device. Using the multi-tube device in FIG. 3B to illustrate, when the catheter connector 448 is disconnected, the catheter tube 452 can extend from one end of the device but the other end may not include a tube. The plug can be inserted into the tubing passageway at that disconnected end to prevent contamination from entering the connector cavity. When a delivery connector 450 is to be connected to the catheter connector 446, the plug can be removed to enable the fluid delivery tube 454 to pass through the tubing passageway at that end of the device 100. It is also contemplated that the plug can be tethered to the device, e.g., similar to the flexible latch 102 described above.

FIG. 4A is a perspective view of an example connector protection device 100 having a padding (e.g., first padding 402 and/or second padding 404) in the internal cavities of the one or more body sections, according to some embodiments of the present disclosure; FIG. 4B is a perspective view of an example connector protection device 100 in a closed configuration, according to some embodiments of the present disclosure. Referring to FIG. 4A, the device can include one or more paddings. For example, the first body section 202 can include a first padding 402 in the first internal cavity 302, and/or the second body section 204 can include a second padding 404 in the second internal cavity 304. The padding 402,404 can be used to help secure the catheter connector 448 and/or the delivery connector 450 within the connector cavity. In other examples, the padding 402,404 can also provide a place to include an antiseptic (e.g., betadine and/or another antiseptic). The antiseptic can be introduced to the first padding 402 and/or the second padding 404 during manufacturing of the device; in other examples, the antiseptic can be added by a health care provider at the point of care. The antiseptic can further prevent infection by preventing contaminants from entering the tubes at the connection site. It is contemplated that the one or more paddings 402,404 can be secured to the respective internal cavity via an adhesive. In other examples, the body sections can include padding tabs to secure the padding. For example, the first padding 402 can be secured within the first internal cavity 302 via one or more first padding tabs 406; the second padding 404 can be secured within the second internal cavity 304 via one or more second padding tabs 408.

FIG. 4A also provides a view of multiple tubes that can be connected before closing the device 100, which is shown in FIG. 4B. The catheter connector 448 can have a catheter tube 452 (or any other type of tube) extending therefrom and through the tubing passageway at one section of the device 100; the fluid delivery connector 450, which can be connected to the catheter connector 448, can include a fluid delivery tube 454 extending therefrom and through the tubing passageway at another section of the device 100. FIG. 4B shows a closed device 100 wherein the catheter tube 452 is extending from one end of the device 100, and the fluid delivery tube 454 is extending from the other end of the device 100. It is contemplated that the devices 100 described herein can be integrated into a system that includes (a) the catheter connector 448 and catheter tube 452, (b) the delivery connector 450 and the fluid delivery tube 454, or (c) both the catheter connector 448, delivery connector 450, and respective tubes. The system can be packaged together and provided to health care professionals in the form of a kit, for example.

In some examples, the device 100 can include a sensor 490. The sensor 490 can be positioned within the first internal cavity 302, the second internal cavity 304 (as shown in FIG. 3A), and/or on an external face of the device 100. The sensor 490 can be a contamination sensor that is used to determine if a contaminant, such as bacteria, is present proximate the connectors. The sensor 490 can be a colorimetric sensor or other sensor that can detect the presence of a contaminant. While some embodiments of the sensor 490 are purely chemical in nature, others comprise an electronic component. Some embodiments of device 100 can include sensors for the purpose of detecting things other than contamination, for example leakage from the tubing, excessive strain, etc.

FIG. 5 is an end perspective view of an example connector protection device 100, according to some embodiments of the present disclosure. The perspective view in FIG. 5 shows who two half circular notches (e.g., first tubing notch 306a and second tubing notch 306b) can form a circular hole 502, serving as a tubing passageway, for the tubing to pass through device 100. As stated above, the view shows how the latch fingers (e.g., first latch finger 106a or any other latch finger) can engage with one or more latch tabs (e.g., first latch tab 206a or any other latch tab).

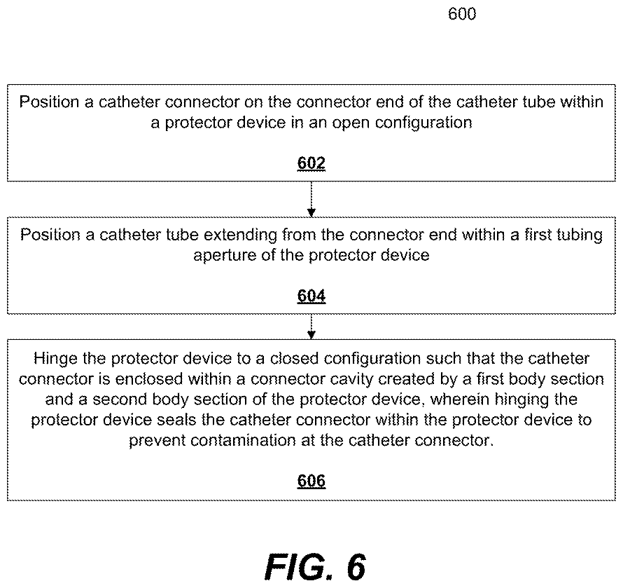

FIG. 6 is a flowchart of an exemplary method 600 for sealing a connector end of a catheter tube, according to some embodiments of the present disclosure. Method 600 can begin with positioning 602 a catheter connector on the connector end of the catheter tube within a connector protection device in an open configuration. The method 600 can include positioning 604 a catheter tube extending from the connector end within a first tubing notch of the connector protection device. The method 600 can include hinging 606 of the connector protection device to a closed configuration such that the catheter connector is enclosed within a connector cavity created by a first body section and a second body section of the connector protection device. Hinging the connector protection device can seal the catheter connector within the connector protection device to prevent contamination at the catheter connector.

The method 600 can end after step 606. In some examples, method 600 can include additional steps according to the present disclosure. For example, method 600 can include attaching a delivery connector to the catheter connector and positioning a fluid delivery tube extending from the delivery connector within a second tubing notch of the connector protection device at an end opposite the first tubing notch. Hinging the connector protection device closed can cause the delivery connector to be sealed within the connector cavity.

It is to be understood that the embodiments and claims disclosed herein are not limited in their application to the details of construction and arrangement of the components set forth in the description and illustrated in the drawings. Rather, the description and the drawings provide examples of the embodiments envisioned. The embodiments and claims disclosed herein are further capable of other embodiments and of being practiced and carried out in various ways. Also, it is to be understood that the phraseology and terminology employed herein are for the purposes of description and should not be regarded as limiting the claims.

Accordingly, those skilled in the art will appreciate that the conception upon which the application and claims are based may be readily utilized as a basis for the design of other structures, methods, and systems for carrying out the several purposes of the embodiments and claims presented in this application. It is important, therefore, that the claims be regarded as including such equivalent constructions.

Furthermore, the purpose of the foregoing Abstract is to enable the United States Patent and Trademark Office and the public generally, and especially including the practitioners in the art who are not familiar with patent and legal terms or phraseology, to determine quickly from a cursory inspection the nature and essence of the technical disclosure of the application. The Abstract is neither intended to define the claims of the application, nor is it intended to be limiting to the scope of the claims in any way. Instead, it is intended that the invention is defined by the claims appended hereto.

1. A tubing connector protection device having an open configuration and a closed configuration, the device comprising:
body sections comprising:
a first body section defining a first internal cavity; and
a second body section defining a second internal cavity, the second body section being hingeably connected to the first body section;
wherein, in the closed configuration, the first internal cavity and the second internal cavity are adjacent to create a connector cavity configured to contain a first connector;
tubing notches comprising:
a first tubing notch positioned at an end of the first body section; and
a second tubing notch positioned at a first end of the second body section;
wherein the first tubing notch and the second tubing notch are positioned to align when the device is in the closed configuration, thereby forming a first hole; and
one or more components selected from the group consisting of a colorimetric sensor, first padding tabs, protective padding, a hinge pin, and a combination thereof;
wherein the first body section comprises the first padding tabs disposed within the first internal cavity and configured to hold a first padding;
wherein the protective padding surrounds the first body section and the second body section on an external face of the respective sections when the device is in a closed configuration; and
wherein the first body section comprises first hinge knuckles, the second body section comprises second hinge knuckles positioned to engage with the first hinge knuckles in an alternating manner, and wherein the hinge pin is configured to engage hinge holes disposed within the first and second hinge knuckles.

2. The device of claim 1, wherein the first hole is located at the first end of the device and is configured to retain tubing for a catheter.

3. The device of claim 1, wherein at least one of the first tubing notch or the second tubing notch comprises a seal configured to close the first hole located at the first end of the device.

4. The device of claim 1 further comprising a plug configured to close the first hole located at the first end of the device.

5. The device of claim 1, wherein the first tubing notch is positioned at a first end of the first body section or the second body section; and
wherein the second tubing notch is positioned at a second end of the second body section or the first body section, the first end being opposite from the second end along a length of the device.

6. The device of claim 1 further comprising:
a third tubing notch positioned at a second end of the first body section; and
a fourth tubing notch positioned at the second end of the second body section;
wherein the third tubing notch and the fourth tubing notch are positioned to align when the device is in the closed configuration to create a second hole.

7. The device of claim 6, wherein:
at least one of the first tubing notch or the second tubing notch comprises a first seal configured to close the first hole at the first end of the device; and
at least one of the third tubing notch or the fourth tubing notch comprises a second seal configured to close the second hole at the second end of the device.

8. The device of claim 6, wherein:
at least one of the first tubing notch or the second tubing notch comprises a first seal configured to close the first hole at the first end of the device; or
at least one of the third tubing notch or the fourth tubing notch comprises a second seal configured to close the second hole at the second end of the device.

9. The device of claim 6 further comprising a plug configured to close at least one of the first hole or the second hole.

10. The device of claim 6 further comprising a first plug configured to close the first hole and a second plug configured to close the second hole.

11. The device of claim 1, wherein the first body section and the second body section form a capsular shape when the device is in the closed configuration.

12. The device of claim 1, wherein:
the first body section comprises a flexible latch;
the second body section comprises a latch peg; and
the flexible latch is configured to detachably connect with the latch peg when the device is in the closed configuration.

13. The device of claim 1, wherein the first body section comprises a latch finger configured to engage with a latch tab disposed on the second body section when the device is in the closed configuration.

14. The device of claim 1, wherein the first body section comprises a plurality of latch fingers, each latch finger configured to engage with one of a plurality of latch tabs disposed on the second body.

15. (canceled)

16. The device of claim 1, wherein the second body section comprises second padding tabs disposed within the second internal cavity and configured to hold a second padding.

17. The device of claim 1, wherein the first padding is a sponge.

18. The device of claim 17, wherein the sponge comprises an antiseptic.

19.-24. (canceled)

25. The device of claim 1, wherein the first body section and the second body section form a single component that are connected via a flexible portion bendable to transition the device from the open configuration and the closed configuration.

26. (canceled)

27. The device of claim 1, wherein at least one of the first body section or the second body section each comprises a seal along an outer periphery of the respective body section, the seal configured to protect the connector cavity when the device is in the closed configuration.

28. A system comprising:
a connector protection device comprising:
a first body section defining a first internal cavity;
a second body section defining a second internal cavity, the second body section being hingeably connected to the first body section;
a first tubing notch positioned at a first end of the connector protection device;
a second tubing notch positioned at a second end of the connector protection device and sized to retain at least a portion of a fluid delivery tube;
a first plug configured to close the first tubing notch; and
a second plug configured to close the second tubing notch; and
a first connector having a first tube extending therefrom;
wherein, when the connector protection device is in a closed configuration, the first internal cavity and the second internal cavity are adjacent to create a connector cavity configured to contain the first connector; and
wherein, when the connector protection device is in a closed configuration, the first tube extends through the first tubing notch.

29. (canceled)

30. The system of claim 28 further comprising a fluid delivery tube comprising a second connector;
wherein the connector cavity is configured to contain a second connector connected to the fluid delivery tube; and
wherein a distance between the first tubing notch and the second tubing notch is substantially the same as a length from a first end of the second connector to a second end of the first connector.

31. (canceled)

32. The system of claim 28, wherein the connector protection device further comprises:
a first seal configured to close the first tubing notch; and
a second seal configured to close the second tubing notch.

33.-35. (canceled)

36. The system of claim 28, wherein the first body section and the second body section form a capsular shape when the device is in the closed configuration.

37.-39. (canceled)

40. The system of claim 28, wherein the first body section comprises a first plurality of padding tabs disposed within the first internal cavity and configured to hold a first padding.

41. The system of claim 40, wherein the second body section comprises a second plurality of padding tabs disposed within the second internal cavity and configured to hold a second padding.

42.-47. (canceled)

48. The system of claim 47, wherein the connector protection device further comprises a contamination sensor being a colorimetric sensor.

49.-55. (canceled)

56. A method for sealing a connector end of a catheter tube, the method comprising:
positioning a first connector on the connector end of the catheter tube within a connector protection device in an open configuration;
positioning a catheter tube extending from the connector end within a first tubing notch of the connector protection device; and
hinging the connector protection device to a closed configuration such that the first connector is enclosed within a connector cavity created by a first body section and a second body section of the connector protection device, wherein hinging the connector protection device seals the first connector within the connector protection device to prevent contamination at the first connector.

57.-63. (canceled)
